(19) United States
(12) Patent Application Publication
(10) Pub. No.: US 2007/0093907 A1
(43) Pub. Date: Apr. 26, 2007

(54) Hydrogel spinal disc implants with swellable articles

(76) Inventors: Dennis W. Goupil, Norcross, GA (US); Bruktawit T. Asfaw, Norcross, GA (US)

Correspondence Address: LAW OFFICE OF COLLEN A. BEARD, LLC, P. O. BOX 1064, DECATUR, GA, 30031-1064, US

(21) Appl. No.: 11/588,057

(22) Filed: Oct. 26, 2006

Related U.S. Application Data
(60) Provisional application No. 60/730,516, filed on Oct. 26, 2005.
(60) Provisional application No. 60/784,723, filed on Mar. 22, 2006.

(51) Int. Cl. A61F 2/44

(52) U.S. Cl. 623/17.16

(57) ABSTRACT

Spinal disc implants containing one or more swellable articles such as dehydrated microspheres.

### CROSS REFERENCE TO RELATED APPLICATIONS

This application is related to and claims priority to U.S. Provisional Application Ser. No. 60/730,516 filed Oct. 26, 2005 and U.S. Provisional Application Ser. No. 60/784,723 filed Mar. 22, 2006, the entire contents of which are incorporated herein by reference.

### BACKGROUND OF THE INVENTION

The spinal disc consists of a soft core called the nucleus pulposus and an outer retaining structure called the annulus fibrosis. The nucleus pulposus and annulus fibrosis are contained between the spinal vertebrae, making intimate contact with the end plates of the vertebrae. The nucleus pulposus is thus bound laterally by the annulus fibrosis and axially by the vertebral body end plates. The nucleus pulposus, annulus fibrosis, and vertebrae are further constrained by the extra-vertebral column structures, e.g. the facet joints. In total, these structures act synergistically to allow motion and axial shock absorption in the spinal column.

The nucleus pulposus consists of a gelatinous composition of proteoglycans, collagen, and water. The water content of the nucleus pulposus ranges from about 90% in the early years to about 40% or less in later years. The presence of water aids in maintaining a hydrostatic pressure in the spinal disc that is necessary for motion and shock absorption. After a person lies prone for several hours, the nucleus pulposus is fully hydrated and the spinal column reaches its maximum height. With activity, the pressure from the upper body causes the disc to lose a small amount of water to the surrounding environment. Thus over a day the disc swells and contracts. This swelling and contraction cycle prevents the build up of pressure in the disc space, protecting the annulus fibrosis from over-pressurization. Another effect of this swelling-contraction cycle is the transportation of nutrients into the disc space.

The nucleus pulposus loses water content as part of the aging process and its ability to provide motion and shock absorption to the spinal column also diminishes. Furthermore, the loss of water may also cause the annulus fibrosis to change from its native concave shape to a shape that places additional stress on the fibers of the annulus. In addition, the loss of water in the nucleus pulposus causes shrinking of the spacing between vertebrae which may cause additional stress on nerve fibers that are attached to the external surface of the annulus fibrosis. This additional stress may result in pain.

The concept of using a swellable synthetic material to replace the aged or damaged nucleus pulposus is taught by, for example, Ray et al. (U.S. Pat. No. 4,772,287), Bao et al. (U.S. Pat. Nos. 5,047,055 and 5,192,326), and Stoy et al. (U.S. Pat. No. 6,726,721). Ray et al. describes an implant that consists of one or two swellable cylinders that are partially dehydrated prior to insertion into the nucleus space through a large incision in the annulus. As the implant swells over several hours, it is ultimately constrained by a porous fabric cover. The implant swells approximately 100% to fill up the nucleus space previously occupied by the native nucleus pulposus that has been surgically resected.

The implant described in U.S. Pat. No. 5,047,055 to Bao et al. is a prefabricated swellable hydrogel that is inserted into the nucleus pulposus through a large incision in the annulus. Unlike the implant described in Ray et al., the device does not include a constraining jacket to contain the polymer implant. The device is typically between about 2 mm and 10 mm in cross-sectional diameter and is rod shaped. The device is implanted in a dehydrated state and is allowed to fully hydrate so that the device is constrained tightly in the cavity formed by the annulus and the end plates. This device swells isotropically due to the lack of a physical constraint, such as an external sack. Because it is rod shaped and a relatively large hole must be created in the annulus for insertion, this device has had some clinical complications associated with expulsion through the weakened annulus.

U.S. Pat. No. 5,192,326 to Bao et al. teaches an implant consisting of hydrogel spheres in an elastic, semi-permeable sack or wrap. The porous wrap, in its unfurled state, has the shape of the nuclear cavity. The hydrogel spheres are at least three times the size of the pores in their swollen state; hence, theoretically not allowing their expulsion through the wrap. The swelling of the device is limited by the wrap. Therefore, swelling is used to fill the space only and does not exert any additional swelling pressure to expand the adjacent vertebrae.

Stoy et al. (U.S. Pat. No. 6,726,721) describes a device claimed to provide axial expansion of the disc space. The device consists of a hydrogel-textile laminate that allows swelling and expansion in the axial direction only. This approach has the theoretical advantages of mitigating expulsion of the implant from the 5 to 7 mm insertion hole as a result of a non-isotropic swelling. This axial swelling may also providing axial lift, which in turn, may separate the adjacent vertebrae enough to reduce strain on nearby nerves. The design of Stoy et al. also has the theoretical advantage of limiting outward stress placed on the annulus that may occur during isotropic swelling. Conversely, this device may not completely fill the space of the vacated nucleus, hence forcing the annulus to remodel to an anatomic shape required to bear additional load.

A swellable nucleus pulposus implant is intended to mimic the native nucleus pulposus, including similar load bearing, space filling and diurnal lift characteristics. It may be difficult, however, to generate enough lifting force using a swellable material alone. The swellable material must be able to imbibe enough water to swell without losing significant strength. It is an object of the present invention to provide a nucleus pulposus implant that swells and provides lift. It is another object of the present invention to provide a nucleus pulposus implant that is not likely to be expelled from the nuclear cavity.

### SUMMARY OF THE INVENTION

The invention is a biomedical implant, especially for use in replacement or augmentation of a spinal disc nucleus pulposus. The implant includes a hydrogel and one or more, preferably a plurality, of swellable articles. The resulting biomedical implant has lift and water uptake properties which make it suitable for use as a spinal disc nucleus pulposus implant. The implant may be formed through in situ formation of the hydrogel with simultaneous delivery of the swellable articles. In a preferred embodiment, the swellable articles are dehydrated, swellable microspheres.

The implant is desirably formed from a composition that is preferably delivered to the implant site as a liquid containing the swellable articles, whereupon the hydrogel precursor forms the hydrogel, entrapping the swellable articles. The articles swell over time, generally due to absorption of fluid.

### DETAILED DESCRIPTION OF THE INVENTION

The invention relates to biomedical implants. More specifically, the invention relates to implants for replacement or augmentation of a spinal disc nucleus pulposus. The invention further relates to methods for augmenting or replacing a spinal disc nucleus pulposus.

The implant includes a hydrogel and one or more, preferably a plurality, of swellable articles embedded therein. “Hydrogel” refers to a material having an aqueous phase with an interlaced polymeric component, with at least 10% to 90% of its weight as water. The hydrogel is desirably formed in situ from a composition that is injectable. The articles are desirably injected with the composition. As the composition forms a hydrogel, the articles are embedded in the hydrogel.

The swellable articles are preferably dehydrated articles that will swell upon taking in fluid—thus increasing the volume of the hydrogel implant over time. The implant will desirably conform in shape to the nucleus space into which it is injected. The implant desirably has a compression modulus of approximately 0.1-5 mega pascals at 10-30% strain, a yield load of approximately 1000-6000 Newtons, a 60-70% strain at failure, and has the ability to withstand cyclic loading under physiologic conditions.

The Hydrogel

The hydrogel can be any of a number of types that are biocompatible and that can be delivered to the spinal disc nucleus pulposus space as a hydrogel precursor and formed into a hydrogel in situ. Generally speaking, the hydrogel precursor is a solution of macromers, monomers, or polymers that can be gelled in response to an initiator.

In a preferred embodiment, the hydrogel is formed from a solution of macromers that are curable, meaning that they can be cured or otherwise modified, in situ, at the tissue site and undergo a phase or chemical change sufficient to retain a desired position and configuration. The hydrogel can be formed from one or more macromers that include a hydrophilic or water soluble region and one or more crosslinkable regions. The macromers may also include other elements such as one or more degradable or biodegradable regions. A variety of factors- primarily the desired characteristics of the formed hydrogel- determines the most appropriate macromers to use. Many macromer systems that form biocompatible hydrogels can be used.

Macromers suitable for use in the compositions described herein are disclosed in WO 01/68721 to BioCure, Inc. Other suitable macromers include those disclosed in U.S. Pat. No. 5,410,016 to Hubbell et al., U.S. Pat. No. 4,938,763 to Dunn et al., U.S. Pat. Nos. 5,100,992 and 4,826,945 to Cohn et al., U.S. Pat. Nos. 4,741,872 and 5,160,745 to De Luca et al., and U.S. Pat. No. 4,511,478 to Nowinski et al.

In a most preferred embodiment, the hydrogel is the hydrogel described in WO 01/68721 to BioCure. This publication discloses a composition useful for tissue bulking that includes macromers having a backbone of a polymer having units with a 1,2-diol and/or 1,3-diol structure. Such polymers include poly(vinyl alcohol) (PVA) and hydrolyzed copolymers of vinyl acetate, for example, copolymers with vinyl chloride, N-vinylpyrrolidone, etc. The backbone polymer contains pendant chains bearing crosslinkable groups and, optionally, other modifiers. The macromers form a hydrogel when crosslinked.

Polyvinyl alcohols (PVAs) that can be used as the macromer backbone include commercially available PVAs, for example Vinol® 107 from Air Products (MW 22,000 to 31,000, 98 to 98.8% hydrolyzed), Polysciences 4397 (MW 25,000, 98.5% hydrolyzed), BF 14 from Chan Chun, Elvanol® 90-50 from DuPont and UF-120 from Unitika. Other producers are, for example, Nippon Gohsei (Gohsenol®), Monsanto (Gelvatol®), Wacker (Polyviol®), Kuraray, Deriki, and Shin-Etsu. In some cases it is advantageous to use Mowiol® products from Hoechst, in particular those of the 3-83, 4-88, 4-98, 6-88, 6-98, 8-88, 8-98, 10-98, 20-98, 26-88, and 40-88 types.

It is also possible to use copolymers of hydrolyzed or partially hydrolyzed vinyl acetate, which are obtainable, for example, as hydrolyzed ethylene-vinyl acetate (EVA), or vinyl chloride-vinyl acetate, N-vinylpyrrolidone-vinyl acetate, and maleic anhydride-vinyl acetate. If the macromer backbones are, for example, copolymers of vinyl acetate and vinylpyrrolidone, it is again possible to use commercially available copolymers, for example the commercial products available under the name Luviskol® from BASF. Particular examples are Luviskol VA 37 HM, Luviskol VA 37 E and Luviskol VA 28. If the macromer backbones are polyvinyl acetates, Mowilith 30 from Hoechst is particularly suitable.

The PVA preferably has a molecular weight of at least about 2,000. As an upper limit, the PVA may have a molecular weight of up to 300,000. Preferably, the PVA has a molecular weight of up to about 130,000, more preferably up to about 60,000, and especially preferably of about 14,000.

The PVA usually has a poly(2-hydroxy)ethylene structure. The PVA may also include hydroxyl groups in the form of 1,2-glycols. The PVA can be a fully hydrolyzed PVA, with all repeating groups being —CH₂—CH(OH), or a partially hydrolyzed PVA with varying proportions (1% to 25%) of pendant ester groups. PVA with pendant ester groups have repeating groups of the structure CH₂—CH(OR) where R is COCH₃ group or longer alkyls, as long as the water solubility of the PVA is preserved. The ester groups can also be substituted by acetaldehyde or butyraldehyde acetals that impart a certain degree of hydrophobicity and strength to the PVA. For an application that requires an oxidatively stable PVA, the commercially available PVA can be broken down by NaIO₄—KMnO₄ oxidation to yield a small molecular weight (2000 to 4000) PVA.

The PVA is prepared by basic or acidic, partial or virtually complete, hydrolysis of polyvinyl acetate. In a preferred embodiment, the PVA comprises less than 50% acetate units, especially less than about 25% of acetate units. Preferred amounts of residual acetate units in the PVA, based on the sum of alcohol units and acetate units, are approximately from 3 to 25%.

The macromers have at least two pendant chains containing groups that can be crosslinked. Group is defmed herein to include single polymerizable moieties, such as acrylates, as well as larger crosslinkable regions, such as oligomeric or polymeric regions. The crosslinkers are desirably present in an amount of from approximately 0.01 to 10 milliequivalents of crosslinker per gram of backbone (meq/g), more desirably about 0.05 to 1.5 milliequivalents per gram (meq/g). The macromers can contain more than one type of crosslinkable group.

The pendant chains are attached via the hydroxyl groups of the backbone. Desirably, the pendant chains having crosslinkable groups are attached via cyclic acetal linkages to the 1,2-diol or 1,3-diol hydroxyl groups. Desirable crosslinkable groups include (meth)acrylamide, (meth)acrylate, styryl, vinyl ester, vinyl ketone, vinyl ethers, etc. Particularly desirable are ethylenically unsaturated fimctional groups. A particularly desirable crosslinker is N-acryloyl-aminoacetaldehyde dimethylacetal (NAAADA) in an amount from about 6 to 21 crosslinkers per macromer.

Specific macromers that are suitable for use in the compositions are disclosed in U.S. Pat. Nos. 5,508,317, 5,665,840, 5,807,927, 5,849,841, 5,932,674, 5,939,489, and 6,011,077.

In one embodiment, units containing a crosslinkable group conform, in particular, to the formula I

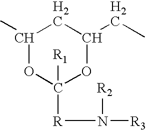

in which R is a linear or branched C₁-C₈ alkylene or a linear or branched C_{1-C}₁₂ alkane. Suitable alkylene examples include octylene, hexylene, pentylene, butylene, propylene, ethylene, methylene, 2-propylene, 2-butylene and 3-pentylene. Preferably lower alkylene R has up to 6 and especially preferably up to 4 carbon atoms. The groups ethylene and butylene are especially preferred. Alkanes include, in particular, methane, ethane, n- or isopropane, n-, sec- or tert-butane, n- or isopentane, hexane, heptane, or octane. Preferred groups contain one to four carbon atoms, in particular one carbon atom.

R₁ is hydrogen, a C₁-C₆ alkyl, or a cycloalkyl, for example, methyl, ethyl, propyl or butyl and R₂ is hydrogen or a C₁-C₆ alkyl, for example, methyl, ethyl, propyl or butyl. R₁ and R₂ are preferably each hydrogen.

R₃ is an olefinically unsaturated electron attracting copolymerizable radical having up to 25 carbon atoms. In one embodiment, R₃ has the structure 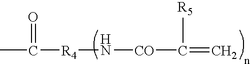
where R₄ is the 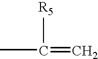
group if n=zero, or the 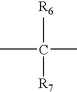
bridge if n=1;

R₅ is hydrogen or C₁-C₄ alkyl, for example, n-butyl, n- or isopropyl, ethyl, or methyl;

n is zero or 1, preferably zero; and

R₆ and R₇, independently of one another, are hydrogen, a linear or branched C₁-C₈ alkyl, aryl or cyclohexyl, for example one of the following: octyl, hexyl, pentyl, butyl, propyl, ethyl, methyl, 2-propyl, 2-butyl or 3-pentyl. R₆ is preferably hydrogen or the CH₃ group, and R₇ is preferably a C₁-C₄ alkyl group. R₆ and R₇ as aryl are preferably phenyl.

In another embodiment, R₃ is an olefmically unsaturated acyl group of formula R₈—CO—, in which R₈ is an olefmically unsaturated copolymerizable group having from 2 to 24 carbon atoms, preferably from 2 to 8 carbon atoms, especially preferably from 2 to 4 carbon atoms. The olefmically unsaturated copolymerizable radical R₈ having from 2 to 24 carbon atoms is preferably alkenyl having from 2 to 24 carbon atoms, especially alkenyl having from 2 to 8 carbon atoms and especially preferably alkenyl having from 2 to 4 carbon atoms, for example ethenyl, 2-propenyl, 3-propenyl, 2-butenyl, hexenyl, octenyl or dodecenyl. The groups ethenyl and 2-propenyl are preferred, so that the group —CO—R₈ is the acyl radical of acrylic or methacrylic acid.

In another embodiment, the group R₃ is a radical of formula
—[CO—NH—(R₉—NH—CO—O)_{q}—R₁₀—O]ₚ—CO—R₈
wherein p and q are zero or one and

R₉ and R₁₀ are each independently lower alkylene having from 2 to 8 carbon atoms, arylene having from 6 to 12 carbon atoms, a saturated divalent cycloaliphatic group having from 6 to 10 carbon atoms, arylenealkylene or alkylenearylene having from 7 to 14 carbon atoms or arylenealkylenearylene having from 13 to 16 carbon atoms, and

R₈ is as defined above.

Lower alkylene R₉ or R₁₀ preferably has from 2 to 6 carbon atoms and is especially straight-chained. Suitable examples include propylene, butylene, hexylene, dimethylethylene and, especially preferably, ethylene.

Arylene R₉ or R₁₀ is preferably phenylene that is unsubstituted or is substituted by lower alkyl or lower alkoxy, especially 1,3-phenylene or 1,4-phenylene or methyl-1,4-phenylene.

A saturated divalent cycloaliphatic group R₉ or R₁₀ is preferably cyclohexylene or cyclohexylene-lower alkylene, for example cyclohexylenemethylene, that is unsubstituted or is substituted by one or more methyl groups, such as, for example, trimethylcyclohexylenemethylene, for example the divalent isophorone radical.

The arylene unit of alkylenearylene or arylenealkylene R₉ or R₁₀ is preferably phenylene, unsubstituted or substituted by lower alkyl or lower alkoxy, and the alkylene unit thereof is preferably lower alkylene, such as methylene or ethylene, especially methylene. Such radicals R₉ or R₁₀ are therefore preferably phenylenemethylene or methylenephenylene.

Arylenealkylenearylene R₉ or R₁₀ is preferably phenylene-lower alkylene-phenylene having up to 4 carbon atoms in the alkylene unit, for example phenyleneethylenephenylene.

The groups R₉ and R₁₀ are each independently preferably lower alkylene having from 2 to 6 carbon atoms, phenylene, unsubstituted or substituted by lower alkyl, cyclohexylene or cyclohexylene-lower alkylene, unsubstituted or substituted by lower alkyl, phenylene-lower alkylene, lower alkylene-phenylene or phenylene-lower alkylene-phenylene.

The group —R₉—NH—CO—O— is present when q is one and absent when q is zero. Macromers in which q is zero are preferred.

The group —CO—NH—(R₉—NH—CO—O)_{q}—R₁₀—O— is present when p is one and absent when p is zero. Macromers in which p is zero are preferred.

In macromers in which p is one, q is preferably zero. Macromers in which p is one, q is zero, and R₁₀ is lower alkylene are especially preferred.

All of the above groups can be monosubstituted or polysubstituted, examples of suitable substituents being the following: C₁-C₄ alkyl, such as methyl, ethyl or propyl, —COOH, —OH, —SH, C₁-C₄ alkoxy (such as methoxy, ethoxy, propoxy, butoxy, or isobutoxy), —NO₂, —NH₂, —NH(C₁-C₄), —NH—CO—NH₂, —N(C₁-C₄ alkyl)₂, phenyl (unsubstituted or substituted by, for example, —OH or halogen, such as Cl, Br or especially I), —S(C₁-C₄ alkyl), a 5- or 6-membered heterocyclic ring, such as, in particular, indole or imidazole, —NH—C(NH)—NH₂, phenoxyphenyl (unsubstituted or substituted by, for example, —OH or halogen, such as Cl, Br or especially I), an olefmic group, such as ethylene or vinyl, and CO—NH—C(NH)—NH₂.

Preferred substituents are lower alkyl, which here, as elsewhere in this description, is preferably C₁-C₄ allyl, C₁-C₄ alkoxy, COOH, SH, —NH₂, —NH(C₁-C₄ alkyl), —N(C₁-C₄ alkyl)₂ or halogen. Particular preference is given to C₁-C₄ alkyl, C₁-C₄ alkoxy, COOH and SH.

For the purposes of this invention, cycloalkyl is, in particular, cycloalkyl, and aryl is, in particular, phenyl, unsubstituted or substituted as described above.

A particularly preferred macromer has a PVA backbone (14 kDa, 17% acetate incorporation) modified with 1.07 meq/g N-acrylamidoacetaldehyde dimethyl acetal (NAAADA) pendant polymerizable groups (about 15 crosslinks per chain). In some preferred embodiments the PVA backbone is also modified with a hydrophobic modifier acetaldehyde diethyl acetal (AADA) present in an amount from about 0 to 4 milliequivalents per gram (meq/g) of PVA (as discussed further below).

Comonomers

WO 01/68721 describes the addition of comonomers to change the characteristics of the hydrogel. Other comonomers are described in WO 06/004940. It may be desirable to include one or more of these comonomers, depending on the desired characteristics of the fmal hydrogel.

Crosslinking Initiators

The ethylenically unsaturated groups of the macromer and any comonomer can be crosslinked via free radical initiated polymerization, including with initiation via photoinitiation, redox initiation, or thermal initiation. Systems employing these means of initiation are well known to those skilled in the art and may be used in the compositions taught herein. The desired amounts of the initiator components will be determined by concerns related to gelation speed, toxicity, extent of gelation desired, and stability.

In one embodiment, a two part redox system is employed. One part of the system contains a reducing agent. Examples of reducing agents are ferrous salts (such as ferrous gluconate dihydrate, ferrous lactate dihydrate, or ferrous acetate), cuprous salts, cerous salts, cobaltous salts, permanganate, manganous salts, and tertiary amines such as N,N,N,N-tetramethylethylene diamine (TMEDA). The other half of the solution contains an oxidizing agent such as hydrogen peroxide, t-butyl hydroperoxide, t-butyl peroxide, benzoyl peroxide, cumyl peroxide, potassium persulfate, or ammonium persulfate.

Either or both of the redox solutions can contain macromer, or it may be in a third solution. The solutions containing reductant and oxidant are combined to initiate the crosslinking. It may be desirable to use a coreductant such as ascorbate, for example, to recycle the reductant and reduce the amount needed. This can reduce the toxicity of a ferrous based system.

Thermal initiation can be accomplished using ammonium persulfate as the crosslinking initiator and optionally using N,N,N,N-tetramethylethylene diamine (TMEDA), which is an amine accelerator.

Modifier Groups

The macromers can include further modifier groups and crosslinkable groups. Some such groups are described in U.S. Pat. Nos. 5,508,317, 5,665,840, 5,807,927, 5,849,841, 5,932,674, 5,939,489, and 6,011,077 and include hydrophobic modifiers such as acetaldehyde diethyl acetal (AADA), butyraldehyde, and acetaldehyde or hydrophilic modifiers such as N-(2,2-dimethoxy-ethyl) succinamic acid, amino acetaldehyde dimethyl acetal, and aminobutyraldehyde dimethyl acetal. These groups may be attached to the macromer backbone, or to other monomeric units included in the backbone. Crosslinkable groups and optional modifier groups can be bonded to the macromer backbone in various ways, for example through a certain percentage of the 1,3-diol units being modified to give a 1,3-dioxane, which contains a crosslinkable group, or a further modifier, in the 2-position. Modifiers include those to modify the hydrophobicity or hydrophilicity, active agents or groups to allow attachment of active agents, photoinitiators, modifiers to enhance or reduce adhesiveness, modifiers to impart thermoresponsiveness, modifiers to impart other types of responsiveness, and additional crosslinking groups.

Attaching a cellular adhesion promoter to the macromers can enhance cellular attachment or adhesiveness of the composition. These agents are well known to those skilled in the art and include carboxymethyl dextran, proteoglycans, collagen, gelatin, glucosaminoglycans, fibronectin, lectins, polycations, and natural or synthetic biological cell adhesion agents such as RGD peptides.

Having pendant ester groups that are substituted by acetaldehyde or butyraldehyde acetals, for example, can increase the hydrophobicity of the macromers and the formed hydrogel. One particularly useful hydrophobic modifying group is acetaldehyde diethyl acetal (AADA) present in an amount from about 0 to 4 milliequivalents per gram (meq/g) of PVA.

Hydrophilic modifiers such as —COOH in the form of N-(2,2-dimethoxy-ethyl) succinamic acid in an amount from about 0 to 2 meq/g PVA can be added to the composition to enhance performance of the composition, such as swelling.

It may also be desirable to include on the macromer a molecule that allows visualization of the formed hydrogel. Examples include dyes and molecules visualizable by magnetic resonance imaging.

Contrast Agents

The implant can be made containing a contrast agent. A contrast agent is a biocompatible material capable of being monitored by, for example, radiography. The contrast agent can be water soluble or water insoluble. Examples of water soluble contrast agents include metrizamide, iopamidol, iothalamate sodium, iodomide sodium, and meglumine. Iodinated liquid contrast agents include Omnipaque®, Visipaque®, and Hypaque-76®. Examples of water insoluble contrast agents are tantalum, tantalum oxide, barium sulfate, gold, tungsten, and platinum. These are commonly available as particles preferably having a size of about 10 μm or less. Coated-fibers, such as tantalum-coated Dacron fibers can also be used.

The contrast agent is incorporated temporarily or permanently in the implant. Both solid and liquid contrast agents can be simply mixed with a solution of the liquid composition prior to crosslinking of the macromers and comonomers. Liquid contrast agent can be mixed at a concentration of about 10 to 80 volume percent, more desirably about 20 to 50 volume percent. Solid contrast agents are desirably included in an amount of about 5 to 40 weight percent, more preferably about 5 to 20 weight percent.

Active Agents

The implant can include an effective amount of one or more biologically or structurally active agents. It may be desirable to deliver the active agent from the formed hydrogel. Active agents that it may be desirable to deliver include prophylactic, therapeutic, diagnostic, and structural agents including organic and inorganic molecules and cells (collectively referred to herein as an “active agent” or “drug”). A wide variety of active agents can be incorporated into the hydrogel. Release of the incorporated additive from the hydrogel is achieved by diffusion of the agent from the hydrogel, degradation of the hydrogel, and/or degradation of a chemical link coupling the agent to the polymer. In this context, an “effective amount” refers to the amount of active agent required to obtain the desired effect.

Examples of active agents that can be incorporated include, but are not limited to, analgesics for the treatment of pain, for example ibuprofen, acetaminophen, and acetylsalicylic acid; antibiotics for the treatment of infection, for example tetracyclines and penicillin and derivatives; and other additives for the treatment of infection, for example silver ions, silver (metallic), and copper (metallic).

Cells and tissue can be incorporated into the composition, including stem cells, autologous nucleus pulposus cells, transplanted autologous nucleus pulposus cells, autologous tissue, fibroblast cells, chondrocyte cells, notochordal cells, allograft tissue and cells, and xenograft tissue and cells.

It may be advantageous to incorporate material of biological origin or biological material derived from synthetic methods of manufacture such as proteins, polypeptides, polysaccharides, proteoglycans, and growth factors.

It may be desirable to include additives to improve the swelling and space-filling properties of the implant in addition to swellable articles as described herein, such as, for example, hydrophilic polymers, such AMPS, etc., or hydrocolloids, such as agar, alginates, carboxymethylcellulose, gelatin, guar gum, gum arabic, pectin, starch, and xanthum gum.

Other additives that may prove advantageous are additives to improve the adhesive properties of the implant, including positively charged polymers, such as Quat, etc., PVA modified with positive-charged moieties attached to the backbone, cyanoacrylates, PVA modified with cyanoacrylate moieties attached to the backbone, chitosan, and mussel-based adhesives.

Incorporation of additives to improve the toughness properties of the injectable disc materials may prove desirable such as low modulus spheres, fibers, etc that act as “crack arrestors” and high modulus spheres, fibers, etc that act as “reinforcing” agents.

Active agents can be incorporated into the composition simply by mixing the agent with the composition prior to administration. The active agent will then be entrapped in the hydrogel that is formed upon administration of the composition. Active agents can be incorporated into preformed articles through encapsulation and other methods known in the art and discussed firther below. The active agent can be in compound form or can be in the form of degradable or nondegradable nano or microspheres. It some cases, it may be possible and desirable to attach the active agent to the macromer or to the preformed article. The active agent may also be coated onto the surface of the preformed article. The active agent may be released from the macromer or hydrogel over time or in response to an environmental condition.

Other Additives

It may be desirable to include a peroxide stabilizer in redox initiated systems. Examples of peroxide stabilizers are Dequest® products from Solutia Inc., such as for example Dequest® 2010 and Dequest® 2060S. These are phosphonates and chelants that offer stabilization of peroxide systems. Dequest® 2060S is diethylenetriamine penta(methylene phosphonic acid). These can be added in amounts as recommended by the manufacturer.

The Swellable Articles

The swellable articles may swell by the absorption of an aqueous fluid from the dried state, lyophilized state, partially-hydrated state, or state where the internal environment is of a higher ionic strength than the surrounding environment. The swellable articles can be made of materials such as polymers, monomers, starches, gums, or poly(amino acids). A nonlimiting list of materials from which the articles can be made is polyvinyl alcohol (PVA), PVA modified with hydrophilic co-monomers, e.g. AMPS, PVA modified with fast crosslinking groups, e.g. NAAADA, PVA modified with polyvinylpyrroline (PVP), polyethylene glycol (PEG), co-polymers of PVA and PEG, polypropylene glycol (PPG), co-polymers of PEG and PPG, co-polymers of PVA and PPG, polyacrylonitrile, hydrocolloids, e.g. agar, alginates, carboxymethylcellulose (CMC), gelatin, etc.

The polymers may be crosslinked, preferably lightly crosslinked hydrophilic polymers. Although these polymers may be non-ionic, cationic, zwitterionic, or anionic, the preferred polymers are cationic or anionic. Especially preferred are acid polymers, which contain a multiplicity of acid fimctional groups, such as carboxylic acid groups, or salts thereof. Examples of such polymers suitable for use herein include those which are prepared from polymerizable, acid-containing monomers, or monomers containing functional groups which can be converted to acid groups after polymerization. Examples of such polymers also include polysaccharide-based polymers such as carboxymethyl starch and cellulose, and poly(amino acid) polymers such as poly(aspartic acid). See US Patent Application 20050065237 to Schmidt et al. for more detail.

Some non-acid monomers may also be included, usually in minor amounts, in preparing the absorbent polymers. Such non-acid monomers include, for example, monomers containing the following types of functional groups: carboxylate or sulfonate esters, hydroxyl groups, amide groups, amino groups, nitrile groups, quaternary ammonium salt groups, and aryl groups (e.g. phenyl groups, such as those derived from styrene monomer). Other potential non-acid monomers include unsaturated hydrocarbons such as ethylene, propylene, 1-butene, butadiene, and isoprene. See U.S. Pat. No. 4,062,817 to Westerman and U.S. Pat. No. 4,076,663 to Masuda et al.

The most preferred non-acid polymer or monomer materials are partially neutralized polyvinyl alcohols that have been modified with an acrylic acid fimctional group.

The swellable articles are preferably dehydrated and swell after implantation in response to fluid uptake. The articles may be fully dehydrated or only partially dehydrated. Upon exposure to liquid (water, saline, body fluids, etc), the spheres will absorb the liquid and swell. In some instances, the design of the device may be to incorporate dehydrated or partially dehydrated articles that swell with sufficient force despite being constrained to provide both space filling and expansion. In other instances, assuming there is sufficient liquid and the dehydrated articles have sufficient room to expand, then the articles will provide space filling only.

As stated above, the articles can swell by other means, including by incorporation of fluid caused by differences in ionic strength between the articles and the polymer.

The amount of swelling can range from 5 to 100 percent, more desirably 5 to 40 percent, most desirably 5 to 20 percent. The time to reach maximum swelling can be designed into the design of the product. In practice, the time to reach maximum swelling can occur within a period of 96 hours, more preferably within a period of 48 hours, and most preferably within a period of 24 hours.

The dehydrated articles may take many forms, such as spheres, particles, fibers, flakes, platelets, disks, or agglomerates. The major limitations in design include swelling capacity, lifting capacity, size, and influence on the viscosity of the injectable formulation.

Swelling capacity, which is the difference between the dehydrated state and the hydrated state, is a function of the chemistry of the article. Swelling in an aqueous environment can be improved by the presence of negatively charged species, such as carboxylic groups. Swelling can also be driven by the electrolyte content of the article. An article with a high salt concentration will draw in liquid in sufficient amount to balance the osmotic pressure between the internal environment of the article and the external liquid. A further way to design the swell factor of the article is by proper choice of the molecular network of the article. For example, a polymer with a highly crosslinked network will swell less than a polymer with a loose network.

Lifting capacity is related to swelling capacity. The major distinction is the amount of force that a dehydrated article may exert on the surrounding tissue. In the case of the nucleus pulposus, a device the merely fills up space in the nucleus cavity is considered to swell, but a device that both fills and expands the adjacent vertebrae is considered to have lift. Like the design of a device that swells, a device that provides lift will absorb the surrounding liquid, but will do so with a higher force.

The size of the swellable article will be chosen largely by the article's swelling or lifting capacity; its size so as to not limit delivery, such as through a needle or delivery catheter; and its effect on the viscosity of the injectable nucleus, such as to not limit delivery though a needle or delivery catheter. As noted above, articles that exhibit a charge can have a significant influence on swelling/lifting, but can also increase the viscosity significantly as well.

The swellable articles may be in the form of microspheres, either compressible or incompressible. The swellable articles may be microspheres made as described in WO 01/68721 to BioCure. The size of a microsphere that may be useful in a synthetic injectable nucleus pulposus ranges from about 0.2 microns to 200 microns, more preferably 0.2 microns to 50 microns, and most preferably about 0.2 microns to 20 microns.

The swellable articles may also be in the shape of fibers. The fiber is described in terms of its major axis, defined as its length, and its minor axis, defmed as its width. Fibers may be flexible, semi-rigid or rigid. A flexible fiber is perceived to be easier to deliver than either a semi-solid or a solid fiber. The minor axis of a flexible fiber that may be useful in the implant ranges from about 0.2 microns to 20 microns, more preferably 0.2 microns to 10 microns, and most preferably about 0.2 microns to 1 micron.

Dehydration of the articles can be achieved through drying, placement in a highly concentrated salt environment, or placement in a nonsolvent environment, such as in an organic solvent for articles that are aqueous based.

Methods of Making the Implant

To make the implant, a liquid composition is prepared by mixing the macromer and any other components such as a crosslinking initiator, in the desired concentrations for each and proportion to each other. The composition may be prepared as a two-part composition, which form the hydrogel when mixed together. In one embodiment, the macromer is formed into a hydrogel prior to implantation. In another embodiment, the macromer is crosslinked into the implant in situ. The swellable articles are desirably mixed in with the liquid macromer prior to implantation and hydrogel formation.

The spinal disc nucleus may have degenerated to the point where denucleation is not required. It may be desirable, however, to denucleate all or a portion of the disc nucleus prior to implantation of the prosthetic nucleus. This can be done by methods known in the field.

In the case of forming the implant prior to administration, a mold may be used to shape the hydrogel or the hydrogel may be free-formed. The liquid composition is placed in a mold, if desired, along with the swellable articles and exposed to conditions to crosslink the macromer. The implant is then implanted into the nucleus, which has been denucleated, if desired. Implantation of the pre-formed implant can be by methods known in the art.

More desirably, the implant is made by in situ crosslinking and hydrogel formation. After denucleation, if desired, an effective amount of the liquid composition containing the swellable articles is placed into the nucleus- preferably by a minimally invasive method. The term “effective amount”, as used herein, means the quantity of composition needed to fill the disc nucleus cavity to the desired level. The composition may be administered over one or a number of treatment sessions.

In the preferred method of making the implant, the liquid composition and swellable articles are drawn up in a 10 ml Luer-lok syringe with care being taken to expel any air bubbles and then delivered using a needle of about 18 Gauge through the small annular access port into the denucleated disc space under fluoroscopic guidance until the disc space has been filled to the desired level. In the case of a two-part composition, the composition is mixed prior to injection in a syringe or using a dual syringe method- transferring the mixture back and forth between two 5 ml syringes using a three way stopcock with care being taken to avoid air bubbles. The composition will preferably crosslink into the formed hydrogel within 5 to 15 minutes post mixing.

The viscosity of the composition is, within wide limits, not critical, but the solution should preferably be a flowable solution that can be injected. In the preferred embodiment, the composition should be injected before substantial crosslinking of the macromers has occurred. This prevents blockage of the syringe needle or catheter with gelled polymer. In addition, in situ crosslinking may allow anchoring of the hydrogel to host tissue by covalently bonding with collagen molecules present within the host tissue.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric.

### EXAMPLE 1

### Yield Load Experiments

Hydrogels were made and tested for their yield load with and without the inclusion of PVA based microspheres. The mechanical testing was conducted by compressing the hydrogel specimens to failure between parallel stainless steel anvils using a Bionix 858 testing machine (MTS Systems Corp., Eden Prairie, Minn.). The specimens were unconstrained laterally. The fixtures were immersed in a phosphate buffered 0.9% saline test bath at 37±1° C. Each specimen was subjected to three conditioning cycles to a nominal strain of 20%. The loading and unloading rate was at 5 mm/min. The time, displacement, and force data were recorded at 10 Hz.

The PVA was Mowiol 3-83 (14 k MW) (from Hoechst Cleanese/Gehring Montgomery). The crosslinker was NAAADA (N-acrylamido acetaldehyde dimethyl acetal) at 15 crosslinkers per chain. Hydrophobic modification of the macromer was accomplished using AADA (acetaldehyde diethyl acetal). Hydrophilic modification of the macromer was accomplished using —COOH. The comonomer DAA (diacetone acrylamide) was included. The initiator was ammonium persulfate and the accelerator was TMEDA (N,N,N,N-tetramethylene diamine). PVA microspheres made as described in sample G of example 2 in WO 01/68720, measuring approximately 50 microns to 100 microns, were dehydrated and then added to the base formulation prior to the addition of TMEDA.

The microspheres were dehydrated as follows. The microspheres were first rinsed with water to remove the saline. They were then stored in acetone for 1 hour, filtered, and equilibrated again in acetone for another hour. They were then equilibrated for 24 hours in acetone, filtered, and dried overnight at 45° C.

The formulation containing the microspheres was poured into a mold and allowed to fully polymerize. The specimen was removed from the mold and placed in saline prior testing on the Bionix 858 testing machine.

| | | AADA | COOH | PVA/ | % | Yield Load |
|---|---|---|---|---|---|---|
| ID | # XL | meq | meq | DAA | microspheres | (N) |
| A | 15 | 2.5 | 1.0 | 1/0.8 | — | 2745 |
| B | 15 | 2.5 | 1.0 | 1/0.8 | 10 | 2237 |

A lower compression yield load was seen with the addition of microspheres.

### EXAMPLE 2

### Lifting Experiments

Hydrogels containing various amounts of PVA based microspheres were made and tested for their lifting capacity. The hydrogels were cured and stored in saline overnight at 37° C. The lifting capacity was then determined by measuring the change in height, weight, and diameter.

The PVA was Mowiol 3-83 (14 k MW). The crosslinker was NAAADA (N-acrylamido acetaldehyde dimethyl acetal) at 15 crosslinkers per chain. Hydrophobic modification of the macromer was accomplished using AADA (acetaldehyde diethyl acetal) at 2.7 milliequivalents. The comonomer DAA (diacetone acrylamide) was included in a 1:1 ratio. The initiator was ammonium persulfate (0.5%) and the accelerator was TMEDA (0.3%). Dehydrated PVA-based microspheres were included, made generally as described in WO 01/68720. The amount of microspheres ranged from 5% percent to 10% by weight. Two types of microspheres were used in the swelling experiments. The microspheres designated as 7-1 contained 1% AMPS, and the microspheres designated as 7-11 contained 11% AMPS.

| | | | | |
|---|---|---|---|---|
| | | % height | % weight | % width |
| ID | % microspheres | change | change | change |

| | | | | | |
|---|---|---|---|---|---|
| A | 5 | (7-1) | 5.97 | 11.16 | 2.26 |
| B | 8.3 | (7-1) | 4.86 | 21.26 | 6.64 |
| C | 10 | (7-1) | 9.03 | 24.47 | 9.84 |
| D | 5 | (7-11) | 7.78 | 15.7 | 2.47 |
| E | 10 | (7-11) | 12.01 | 22.64 | 5.02 |

### EXAMPLE 3

### Effect of Microspheres Addition in Formulations

The PVA was Mowiol 3-83 (14 k MW). The crosslinker was NAAADA (N-acrylamido acetaldehyde dimethyl acetal) at 15 crosslinkers per chain. Hydrophobic modification of the macromer was accomplished using AADA (acetaldehyde diethyl acetal) at 2.7 milliequivalents. The comonomer DAA (diacetone acrylamide) was included in a 1 to 1 ratio. 0.25% APS was dissolved in the formulation by stirring for 1 min. 0.3% TMEDA was added to the formulation and stirred for 15 sec. The dry microspheres were added and mixed for 20 sec. The formulation was poured into molds and allowed to cure for 10 min. Polymers were demolded, weighed, and their height and diameter measured. The polymers were then stored in 50 ml saline and placed in a 37° C. oven for the time indicated.

| | T = 0 | | | T = 24 hr | | | T = 72 hrs | | | % Increase after | | |
|---|---|---|---|---|---|---|---|---|---|---|---|---|
| | Wt | Ht | D | Wt | Ht | D | Wt | Ht | D | 72 hrs | | |
| % ms | (g) | (mm) | (mm) | (g) | (mm) | (mm) | (g) | (mm) | (mm) | Wt | Ht | D |
| 2 | 2.894 | 7.99 | 21.46 | 3.065 | 8.30 | 21.98 | 3.024 | 8.15 | 21.90 | 4.49 | 2.00 | 2.05 |
| 4 | 2.879 | 7.87 | 21.25 | 3.069 | 8.36 | 21.75 | 3.063 | 8.22 | 21.76 | 6.50 | 4.44 | 2.40 |
| 6 | 2.896 | 7.90 | 21.15 | 3.155 | 8.24 | 21.88 | 3.146 | 8.19 | 22.00 | 8.63 | 3.67 | 4.01 |
| 8 | 2.915 | 7.79 | 21.24 | 3.175 | 8.05 | 21.88 | 3.191 | 8.21 | 21.90 | 9.46 | 5.39 | 3.11 |
| 10 | 2.902 | 7.81 | 21.39 | 3.240 | 8.18 | 22.26 | 3.305 | 8.19 | 22.25 | 13.88 | 4.86 | 4.02 |
| 20 | 2.897 | 8.07 | 21.92 | 3.202 | 8.30 | 22.27 | 3.320 | 8.37 | 22.47 | 14.6 | 3.71 | 2.50 |

The presence of dehydrated microspheres can enhance the swelling properties of implants. The addition of 5 percent to 10 percent by weight microspheres increases the height of specimens by approximately 5 percent to 12 percent and the width by approximately 2 percent to approximately 10 percent. This range of swelling may be sufficient to fill the nucleus space without adding additional stress to the annulus. Different specimen shapes, such as may occur after an injection of a polymer nucleus pulposus, may swell in different ways. The addition of approximately 10 percent microspheres in an implant does not significantly alter the yield load of the specimen.

### EXAMPLE 4

### Swellable Fibers A

PVA (mw-14,000) was modified with 1.07 mmol/g of NAAADA and 2.7 mmol/g of AADA. 20 g of comonomer DAA was slowly dissolved in 20 g of a 24% PVA solution. 0.25 g of ammonium persulphate was dissolved in 4.9 g of the resulting solution. 15 ul of TMEDA was added and mixed for 20 sec. 0.1 g of Poly(isobutylene-co-maleic acid) (2%), sodium salt fibers (24-40 um in diameter) were added and mixed for 20 sec, then delivered into a disc mold, resulting in a polymer. The cured polymer was then stored in saline for 24 hrs at 37° C. The percent height change was 11.29; the percent weight change was 20.79; and the percent width change was 4.64.

### EXAMPLE 5

### Swellable Fibers B

PVA (mw=14,000) was modified with 1.07 mmol/g of NAAADA as crosslinker and 2.7 mmol/g of AADA. 20 g of comonomer DAA was slowly dissolved in 20 g of a 24% PVA solution. 0.25 g of ammonium persulphate was dissolved in 4.5 g of the resulting solution. 15 ul of TMEDA was added and mixed for 20 sec. 0.5 g of poly(isobutylene-co-maleic acid) (10%), sodium salt fibers (24-40 um in diameter) were added and mixed for 20 sec, then delivered into a disc mold, resulting in a polymer. The cured polymer was then stored in saline for 24 hrs at 37° C. The percent height change was 36.2; the percent weight change was 104.3; and the percent width change was 22.8.

### EXAMPLE 6

### Sephadex Microspheres A

PVA (mw=14,000) was modified with 1.07 mmol/g of NAAADA as crosslinker and 2.7 mmol/g of AADA. 20 g of comonomer DAA was slowly dissolved in 20 g of a 24% PVA solution. 0.25 g of ammonium persulphate was dissolved in 4.8 g of the resulting solution. 15 ul of TMEDA was added and mixed for 20 sec. 0.2 g of Sephadex G-25 (4%) (20-50 um in diameter) were added and mixed for 20 sec, then delivered into a disc mold, resulting in a polymer. The cured polymer was then stored in saline for 24 hrs at 37° C. The percent height change was 3.24; the percent weight change was 6.03; and the percent width change was 2.12.

### EXAMPLE 7

### Sephadex Microspheres B

PVA (mw=14,000) was modified with 1.07 mmol/g of NAAADA as crosslinker and 2.7 mmol/g of AADA. 20 g of comonomer DAA was slowly dissolved in 20 g of a 24% PVA solution. 0.25 g of ammonium persulphate was dissolved in 4.8 g of the resulting solution. 15 ul of TMEDA was added and mixed for 20 sec. 0.5 g of Sephadex G-25 (10%) (20-50 um in diameter) were added and mixed for 20 sec, then delivered into a disc mold, resulting in a polymer. The cured polymer was then stored in saline for 24hrs at 37° C. The percent height change was 6.53; the percent weight change was 11.07; and the percent width change was 3.33.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A spinal disc nucleus pulposus implant comprising a hydrogel and one or more swellable articles.

2. The implant of claim 1 wherein the swellable articles are dehydrated articles.

3. The implant of claim 1, wherein the swellable articles are dehydrated microspheres.

4. The implant of claim 1, wherein the swellable articles are embedded in the hydrogel.

5. The implant of claim 1, wherein the swellable articles swell in response to the absorption of fluid and provide lift.

6. The implant of claim 1, wherein the swellable articles expand so that the implant can fill a nucleus pulposus cavity.

7. The implant of claim 1, wherein the swellable articles swell with sufficient force so that the implant provides expansion of a spinal disc when the implant is implanted into the nucleus pulposus cavity.

8. The implant of claim 1, wherein the swellable articles are present in an amount of from about 1 to 10 weight percent.

9. The implant of claim 1, wherein the swellable articles swell from about 5 to 20 percent.

10. The implant of claim 1, wherein the swellable articles reach maximum size after swelling for about 24 hours.

11. The implant of claim 3, wherein the microspheres range in size from about 0.2 microns to 200 microns.

12. The implant of claim 3, wherein the hydrogel and microspheres are PVA based.

13. The implant of claim 1, wherein the swellable articles swell due to a difference in ionic strength between the articles and the surrounding environment.
